(19) United States
(12) Patent Application Publication
(10) Pub. No.: US 2013/0112152 A1
(43) Pub. Date: May 9, 2013

(54) SNP Alleles Associated with Leopard Complex Spotting and Congenital Stationary Blindness and Agents, Methods and Kits Thereof

(71) Applicants: Rebecca Bellone, Tampa, FL (US); Sheila Archer, Quill Lake (CA); Claire Wade, Como (AU); Bruce Grahn, Saskatoon (CA); Lynne Sandmeyer, Saskatoon (CA); George Forsyth, Aberdeen (CA)

(72) Inventors: Rebecca Bellone, Tampa, FL (US); Sheila Archer, Quill Lake (CA); Claire Wade, Como (AU); Bruce Grahn, Saskatoon (CA); Lynne Sandmeyer, Saskatoon (CA); George Forsyth, Aberdeen (CA)

(21) Appl. No.: 13/292,688

(22) Filed: Nov. 9, 2011

(51) Int. Cl. C12Q 1/68; A01K 67/00

(52) U.S. Cl. CPC C12Q 1/6883; A01K 67/00
USPC 119/712; 435/6.11; 506/9; 536/24.31; 506/16; 536/24.33

(57) ABSTRACT

The present disclosure relates to single nucleotide polymorphisms and primers and probes useful for screening for, diagnosing or detecting congenital stationary night blindness or for selecting or detecting horse coat patterns.

### FIELD

The application relates to methods, agents and kits for screening, detecting or diagnosing congenital stationary night blindness. Further, the application relates to methods, agents and kits for screening or detecting horse coat patterns.

### BACKGROUND

Coat color has been a fascinating topic of genetic discussion and discovery for over a century. The pigment genes of mice were one of the first genetic systems to be explored through breeding and transgenic studies. To date at least 127 loci involved in pigmentation have been described (Silvers, 1979; Bennett and Lamoreux, 2003). Often the genes that affect pigmentation in the skin and hair influence other body systems, and many such genes have been studied in several different mammals. One of the many extensively studied examples is oculocutaneous albinism type 1; a developmental disorder in humans that affects pigmentation in the skin as well as eye development. This disease is caused by mutations in the tyrosinase gene (TYR), which is involved in the first step of melanin production (Toyofuko et al. 2001; Ray et al. 2007).

Horses (Equus Caballus) are among the domesticated animals valued by breeders and enthusiasts for their variety and beauty of coat color and patterns. The genetic mechanisms involved in several different variations of coloration and patterning in horses have been reported including; chestnut, frame overo, cream, black, silver dapple, sabino-1 spotting, tobiano spotting and dominant white spotting (Marklund et al. 1996; Metallinos et al. 1998; Mariat et al. 2003; Reider et al. 2003; Brunberg et al. 2006; Brooks and Bailey 2005; Brooks et al., 2007; Haase et al. 2007). Although there are several inherited ocular diseases reported in the horse (cataracts, glaucoma, anterior segment dysgenesis, and congenital stationary night blindness), the causative genetic mutations and the pathogenesis of some of these ocular disorders remain unknown.

Appaloosa spotting is characterized by patches of white in the coat that tend to be symmetrical and centered over the hips. In addition to the patterning in the coat, appaloosa spotted horses have three additional pigmentation traits; striped hooves, readily visible nonpigmented sclera around the eye, and mottled pigmentation around the anus, genitalia, and muzzle (Sponenberg and Beaver 1983). The extent of spotting varies widely among individuals, resulting in a collection of patterns which are termed the “leopard complex” (Sponenberg et al. 1990). This variation encompasses a broad spectrum of patterns; including those possessing very minimal patches on the rump (known as a “lace blanket”), a white body with many oval or round pigmented spots dispersed throughout (known as “leopard”, from which the genetic locus is named), as well as a nearly complete depigmentation (known as “fewspot”) (FIG. 1). A single autosomal dominant gene, Leopard Complex (LP), is thought to be responsible for the inheritance of these patterns and associated traits, while modifier genes are thought to play a role in determining the amount of white patterning that is inherited (Miller 1965; Sponenberg et al. 1990; Sponenberg et al. 2009). Horses that are homozygous for appaloosa spotting (LP/LP) tend to have fewer spots of pigment in the white patterned areas; these horses are known as “fewspots” (largely white body with little to no spots) and “snowcaps” (white over the croup and hips with little to no spots) (Sponenberg et al. 1990; Lapp & Carr 1998) (FIG. 1).

A whole genome scanning panel of microsatellite markers was used to map LP to a 6 cM region on ECA1 (Terry et al. 2004). Prior to the sequencing of the equine genome, two candidate genes Transient Receptor Potential Cation Channel, Subfamily M, Member 1 (TRPM1) and Oculoctaneous Albinism Type II (OCA2) were suggested based on comparative phenotypes in humans and mice (Terry et al. 2004). Both TRPM1 and OCA2 were FISH mapped to ECA1, to the same interval as LP (Bellone et al. 2006a). One SNP in the equine OCA2 gene has been ruled out as the cause for appaloosa spotting (Bellone et al. 2006b).

TRPM1, also known as Melastatin 1 (MLSN1), is a member of the transient receptor potential (TRP) channel family. Channels in the TRP family may permit Ca²⁺ entry into hyperpolarized cells, producing intracellular responses linked to the phosphatidylinositol and protein kinase C signal transduction pathways (Clapham et al. 2001). TRPs are important in cellular and somatosensory perception (Nilius, 2007). Defects in a light-gaited TRP channel results in a loss of phototransduction in Drosophila (reviewed in Kim, 2004). Although the specific function of TRPM1 in melanogenesis has yet to be described, cellular sensation and intercellular signaling is vital for normal melanocyte migration (reviewed in Steingrimsson et al. 2006). In mice and humans, the promoter region of this gene contains four consensus binding sites for a melanocyte transcription factor, MITF (Hunter et al. 1998; Zhiqi et al. 2004). One of these sites, termed an M-box, is unique to melanocytic expression (Hunter et al. 1998). TRPM1 is downregulated in highly metastatic melanoma cells, suggesting that this protein plays an important role in normal melanogenesis (Duncan et al. 1998).

In humans TRPM1 is expressed in several isoforms (Xu et al. 2001: Fang and Setaluri 2000). The long isoform, termed MLSN-L, is thought to be responsible for Ca²⁺ influx (Xu et al. 2001). It is possible the large relative expression difference that was detected for the long isoform of TRPM1 may interfere with Ca²⁺ signaling in the melanocytes and thus participate in the biological mechanisms of appaloosa spotting (Bellone et al., 2008).

The specific function of TRPM1 in melanocytes is unknown. It has been described as a tumor suppressor that may regulate the metastatic potential of melanomas, as its expression declines with increased metastatic potential (Duncan et al. 1998; Deeds et al. 2000; Duncan et al. 2001). Treatment of pigmented melanoma cells with a differentiation inducing agent upregulated the long isoform of this gene (Fang and Setaluri, 2000). TRPM1 therefore has potential roles in Ca²⁺-dependent signaling related to melanocyte proliferation, differentiation, and/or survival.

One potential role of TRPM1 in melanocyte survival is in interaction with the signaling pathway of the cell surface tyrosine kinase receptor, KIT, and its ligand KITLG. Signaling through the KIT receptor is critical for the growth, survival and migration of melanocyte precursors (reviewed by Erikson, 1993). It has been shown that both phospholipase C activation and Ca²⁺ influx are important in supporting KIT-positive cells (Berger 2006). Stimulation with KIT ligand while blocking Ca²⁺ influx led to a novel form of cell death that is termed activation enhanced cell death (AECD) (Gommerman and Berger 1998). It is possible that during melanocyte proliferation and differentiation, when KIT positive cells are being stimulated by the ligand in vivo, the absence of TRPM1 expression may result in decreased Ca²⁺ influx and ultimately result in AECD. Early melanocyte death could explain LP hypopigmentation patterns.

An association of homozygosity for LP and congenital stationary night blindness (CSNB) has been documented (Sandmeyer et al. 2007). CSNB is characterized by a congenital and non-progressive scotopic visual deficit (Witzel et al. 1977, 1978; Rebhun et al. 1984). Affected horses may exhibit apprehension in dimly lit conditions and may be difficult to train and handle in phototopic (light) and scotopic (dark) conditions (Witzel et al. 1977, 1978; Rebhun et al. 1984). Affected animals occasionally manifest a bilateral dorsomedial strabismus (improper eye alignment) and nystagmus (involuntary eye movement) (Rebhun et al. 1984; Sandmeyer et al. 2007). CSNB is diagnosed by an absent b-wave and a depolarizing a-wave in scotopic (dark-adapted) electroretinography (ERG) (FIG. 2). This ERG pattern is known as a “negative ERG” (Witzel et al. 1977). No morphological or ultrastructural abnormalities have been detected in the retinas of horses with CSNB (Witzel et al. 1977; Sandmeyer et al. 2007). A similar “negative ERG” is seen in the Schubert-Bornshein type of human CSNB (Schubert and Bornshein 1952; Witzel et al. 1978). This type of CSNB is thought to be caused by a defective neural transmission within the retinal rod pathway (Witzel et al. 1977, 1978; Sandmeyer et al. 2007). Rod photoreceptors are most sensitive under scotopic conditions. In the dark, these cells exist in a depolarized state. They hyperpolarize in response to light, and signaling occurs through reductions in glutamate release (Stryer 1991). This hyperpolarization is responsible for the a-wave of the electroretinogram. Normally this results in stimulation of a population of bipolar cells, the ON bipolar cells. The glutamate receptor of the ON bipolar cells is a metabotropic glutamate receptor (MGluR6) and this receptor is expressed only in the retinal bipolar cell layer (Nomura et al. 1994; Nakanishi et al. 1998). The MGluR6 receptors sense the reduction in synaptic glutamate and produce a response that depolarizes the ON bipolar cell (Nakanishi et al. 1998). This depolarization is responsible for the b-wave of the electroretinogram. The ERG characteristics of the Schubert-Bornshein type of CSNB are consistent with a failure in depolarization of the ON bipolar cell (Sandmeyer et al. 2007).

Decreased expression of TRPM1 has been implicated as the cause for both LP and CSNB (Bellone et al. 2008 and WO/2009/105890). Furthermore, this decreased expression in the horse led others to investigate the role of TRPM1 in on bipolar cell signalling and in human CSNB. Recently TRPM1 has been shown to be the cation channel closed in response to signalling through MGluR6 and the cause of several forms of human CSNB Bellone et al. 2008; Shen et al. 2009; Morgans et al. 2009; Van Genderen et al. 2009; Audo et al. 2009; Nakamura et al. 2010; Li et al. 2009). LP and CSNB have been fine-mapped in the horse to a 173-kb haplotype on ECA1 and Illumina sequencing identified SNPs in the horse for further investigation (Bellone et al. 2010).

### SUMMARY

The inventors have identified three single nucleotide polymorphisms (SNPs) that show complete association with Leopard Complex (LP/LP or LP) and CSNB in a panel of 552 horses.

Accordingly, the present disclosure provides a method of screening for, detecting or diagnosing congenital stationary night blindness (CSNB) in a horse comprising determining the presence of at least one SNP allele associated with CSNB, wherein the at least one SNP allele associated with CSNB is (a) located at position 108281765 of ECA1 (horse chromosome 1) and the allele associated with CSNB is C (or G if the complementary strand); (b) located at position 108288853 of ECA1 (horse chromosome 1) and the allele associated with CSNB is T (or A if the complementary strand); or (c) located at position 108337089 of ECA1 (horse chromosome 1) and the allele associated with CSNB is G (or C if the complementary strand). In an embodiment, the method comprises determining the presence of at least two of the SNP alleles associated with CSNB. In another embodiment, the method comprises determining the presence of all three of the SNP alleles associated with CSNB.

In an embodiment, the method further comprises managing the CSNB in the horse if two copies of the allele associated with CSNB are present, i.e. the subject is homozygous for the associated allele. In an embodiment, the method further comprises treating the horse for CSNB if two copies of the allele associated with CSNB are present, i.e. the subject is homozygous for the associated allele.

In one embodiment, the horse is an Appaloosa horse, American Miniature Horse, British Spotted Pony, Pony of the Americas or a Knabstrupper horse.

In another aspect, the present disclosure provides a method of selecting or detecting coat patterns or leopard complex spotting (LP, also referred to as appaloosa spotting) in a horse comprising determining the presence of at least one SNP allele associated with LP, wherein the at least one SNP allele associated with LP is (a) located at position 108281765 of ECA1 (horse chromosome 1) and the allele associated with LP is C (or G if the complementary strand); (b) located at position 108288853 of ECA1 (horse chromosome 1) and the allele associated with LP is T (or A if the complementary strand); or (c) located at position 108337089 of ECA1 (horse chromosome 1) and the allele associated with LP is G (or C if the complementary strand). In an embodiment, the method comprises determining the presence of at least two of the SNP alleles associated with LP. In another embodiment, the method comprises determining the presence of all three of the SNP alleles associated with LP.

In one embodiment, the methods for detecting or selecting coat patterns in a horse further comprise selecting a horse that has no allele associated with LP and selecting a horse that has two copies of an allele associated with LP, breeding the selected horses together to generate a horse with an LP/lp genotype. In yet another embodiment, the methods further comprise selecting two horses, each with two copies of an allele associated with LP and breeding the selected horses together to generate a horse with an LP/LP genotype. In, yet a further embodiment, the methods further comprise selecting two horses, each with no alleles associated with LP and breeding the selected horses together to generate a horse with an lp/lp genotype or selecting a single horse with no allele associated with LP and breeding a selected horse with a thoroughbred, quarter horse, or other lp/lp horse to generate a horse with an lp/lp genotype. In yet another embodiment, the methods further comprise selecting two horses, each with one allele associated with LP and breeding the selected horses to generate a horse with an LP/LP, LP/lp or lp/lp genotype. In yet another embodiment, the methods further comprise selecting two horses, the first with one allele associated with LP and the second with two alleles associated with LP and breeding the selected horses to generate a horse with an LP/LP or LP/lp genotype. In yet a further embodiment, the methods further comprise selecting two horses, the first with one allele associated with LP and the second with no alleles associated with LP and breeding the selected horses to generate a horse with an LP/lp or lp/lp genotype. In one embodiment, the horse is an Appaloosa. In another embodiment, the horse is a Knabstrupper. In yet another embodiment, the horse is an American Miniature. In a further embodiment, the horse is a British Spotted Pony. In yet a further embodiment, the horse is a Pony of the Americas.

Further, the disclosure provides compositions and kits that can be used to select or detect coat patterns or LP or to diagnose or detect CSNB.

Other features and advantages of the present disclosure will become apparent from the following detailed description. It should be understood, however, that the detailed description and the specific examples while indicating embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The invention will now be described in relation to the drawings in which:

FIG. 1 is a series of photographs of horses displaying different appaloosa coat color patterns: (a) lace blanket (LP/lp); (b) spotted blanket (LP/lp); (c) leopard (LP/lp); (d) snowcap blanket (LP/LP); and (e) fewspot (LP/LP).

FIG. 2 is a scotopic electroretinogram from an lp/lp Appaloosa (left) and an LP/LP Appaloosa with CSNB (right). Note the absence of a b-wave in the ERG tracing from the LP/LP horse. (50 msec, 100 mV).

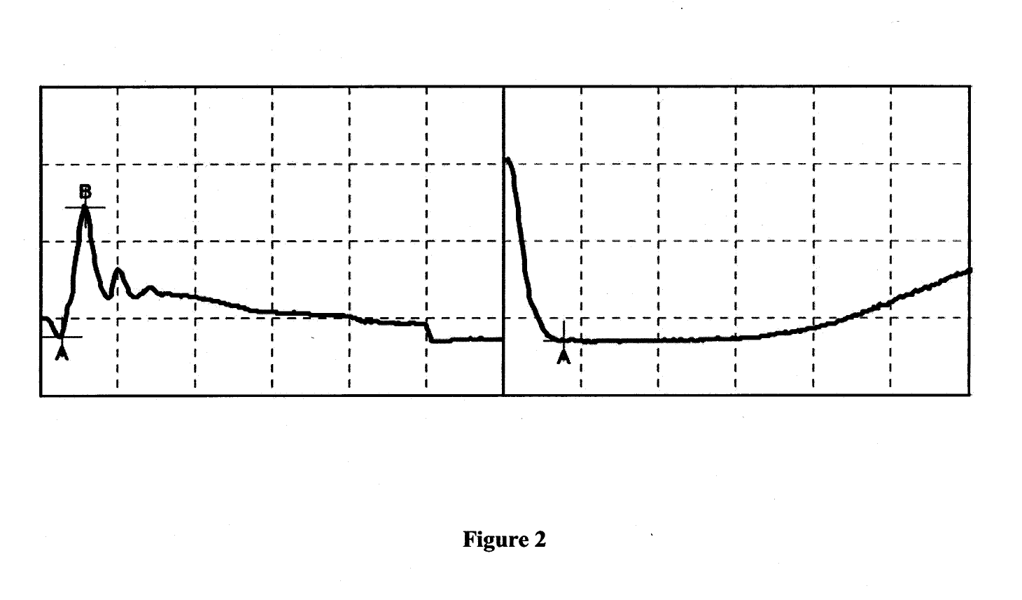

### DETAILED DESCRIPTION

### (A) Single Nucleotide Polymorphisms

The present inventors have identified three single nucleotide polymorphisms associated with congenital stationary night blindness (CSNB) and horse coat patterns (LP genotype) in the TRPM1 gene. In one embodiment, the single nucleotide polymorphism is located at position 108281765 of ECA1 (horse chromosome 1) and the allele associated with CSNB or LP is C. In another embodiment, the single nucleotide polymorphism is located at position 108288853 of ECA1 (horse chromosome 1) and the allele associated with CSNB or LP is T. In yet another embodiment, the single nucleotide polymorphism is located at position 108337089 of ECA1 (horse chromosome 1) and the allele associated with CSNB or LP is G. It is to be understood that wherever the associated allele, C, T or G, respectively, is described herein, it includes the complementary allele G, A or C, respectively. Thus for the SNP at position 108281765 of ECA1, the associated allele is C or if looking at the complementary strand is G. For the SNP at position 108288853 of ECA1, the associated allele is T or if looking at the complementary strand is A. For the SNP at position 108337089 of ECA1, the associated allele is G or if looking at the complementary strand is C.

The term “single nucleotide polymorphism” or “SNP” as used herein refers to a genetic variation in the DNA sequence that occurs at a single nucleotide position.

The term “ECA1” as used herein refers to horse chromosome 1.

The term “TRPM1 gene” refers to the gene encoding the transient receptor potential cation channel, subfamily M member 1 and includes, without limitation, all known TRPM1 genes, including naturally occurring variants, and including those deposited in Genbank with accession number XM_{—}001492235.1 and accession number NM_{—}002420.

### (B) Methods

### (i) Definitions

The term “horse” as used herein includes all breeds, including, without limitation, Appaloosa, Noriker, Knabstrupper, American Miniature, Pony of the America, Australian spotted pony, British spotted pony, Altai, Mongolian Pony, Colorado Ranger Horse, Falabella, Spanish Mustang, and Karabaier or any other LP carrying breed. In one embodiment, the breed is Appaloosa. In another embodiment, the breed is Knabstrupper. In yet another embodiment, the breed is American Miniature. In a further embodiment, the breed is a British Spotted Pony. In yet a further embodiment, the breed is a Pony of the Americas.

The term “sample” as used herein refers to any fluid, cell or tissue sample from a subject, such as a horse, in which DNA can be isolated. In one embodiment, the sample is a blood sample. In another embodiment, the sample is a hair sample. In yet another embodiment, the sample is a skin sample. If the horse has a variation in skin pigmentation, then the skin sample can be pigmented or unpigmented skin. In a further embodiment, the sample is hair. In an additional embodiment, the sample is blood or serum. The methods also contemplate prenatal screening, for example, the sample can be from a fetus of a horse.

### (ii) Methods to Screen for, Diagnose or Detect Congenital Stationary Night Blindness

In one aspect, the disclosure provides a method of screening for, diagnosing or detecting congenital stationary night blindness (CSNB) in a horse comprising determining the presence of at least one SNP associated with CSNB, wherein the at least one SNP allele associated with CSNB is (a) located at position 108281765 of ECA1 (horse chromosome 1) and the allele associated with CSNB is C; (b) located at position 108288853 of ECA1 (horse chromosome 1) and the allele associated with CSNB is T; or (c) located at position 108337089 of ECA1 (horse chromosome 1) and the allele associated with CSNB is G. As disclosed herein, it is to be understood that wherever the associated allele, C, T or G, respectively, is described herein, it includes the complementary allele G, A or C, respectively. Thus for the SNP at position 108281765 of ECA1, the associated allele is C or if looking at the complementary strand is G. For the SNP at position 108288853 of ECA1, the associated allele is T or if looking at the complementary strand is A. For the SNP at position 108337089 of ECA1, the associated allele is G or if looking at the complementary strand is C.

The phrase “screening for, diagnosing or detecting congenital stationary night blindness” refers to a method or process of determining if an individual has or does not have congenital stationary night blindness, and includes determining the grade or severity of congenital stationary night blindness.

The term “congenital stationary night blindness” or “CSNB” as used herein refers to a non-progressive, inherited retinal disorder that is characterized by night blindness, decreased visual acuity, myopia, nystagmus and strabismus. It is diagnosed by an absent b-wave and depolarizing a-wave on an electroretinograph (ERG). The term also includes the Schubert-Bornshein type of human congenital stationary night blindness.

The phrase “determining the presence of at least one SNP allele associated with CSNB” as used herein includes, without limitation, obtaining a sample and testing the sample for hybridization with a probe that hybridizes to the SNP allele or obtaining a sample, amplifying from the sample a region surrounding the SNP allele and analyzing the amplified region for the nucleotide at the SNP position. Analysis of the amplified region for the nucleotide at the SNP position, includes, without limitation, sequencing the region, or subjecting the amplified region to any SNP genotyping assay such as a restriction fragment length polymorphism among others.

One SNP genotyping assay is a Custom TaqMan® SNP Genotyping Assay which involves two primers and two probes. Primers correspond to the region of DNA amplified during a polymerase chain reaction. Each probe is completely complementary to only one allele and each probe is labeled with a different reporter fluorophore. Therefore alleles are determined by fluorescence detected (see Table 2 as an example).

In an embodiment, the method further comprises managing the CSNB in the horse if two copies of the allele associated with CSNB are present, i.e. the subject is homozygous for the associated allele. In an embodiment, the method further comprises treating the horse for CSNB if two copies of the allele associated with CSNB are present, i.e. the subject is homozygous for the associated allele.

The phrase “managing the CSNB in the horse” as used herein refers to managing strategies to minimize the risk of injury to both the horse and the owner/handler, including without limitation, installing night lighting in paddock or in shelter and stalls, using proper fencing such as solid or break away materials designed to minimize injuries, and pasturing with non-aggressive companion animals.

It is contemplated that the methods described herein can be used in combination with other methods of screening for, diagnosing or detecting congenital stationary night blindness. For example, the method can be used in combination with determining the genotype for LP of the horse.

### (iii) Methods to Detect or Select Coat Patterns in Horses

Predicting the genotype of LP from a phenotype can be difficult because of the variability in pattern and complications when other spotting patterns caused by different loci are present. Accordingly, in another aspect, the disclosure provides a method of detecting or selecting different coat patterns in a horse comprising determining the presence of at least one SNP allele associated with LP, wherein the at least one SNP allele associated with LP is (a) located at position 108281765 of ECA1 (horse chromosome 1) and the allele associated with LP is C; (b) located at position 108288853 of ECA1 (horse chromosome 1) and the allele associated with LP is T; or (c) located at position 108337089 of ECA1 (horse chromosome 1) and the allele associated with LP is G. As disclosed herein, it is to be understood that wherever the associated allele, C, T or G, respectively, is described herein, it includes the complementary allele G, A or C, respectively. Thus for the SNP at position 108281765 of ECA1, the associated allele is C or if looking at the complementary strand is G. For the SNP at position 108288853 of ECA1, the associated allele is T or if looking at the complementary strand is A. For the SNP at position 108337089 of ECA1, the associated allele is G or if looking at the complementary strand is C.

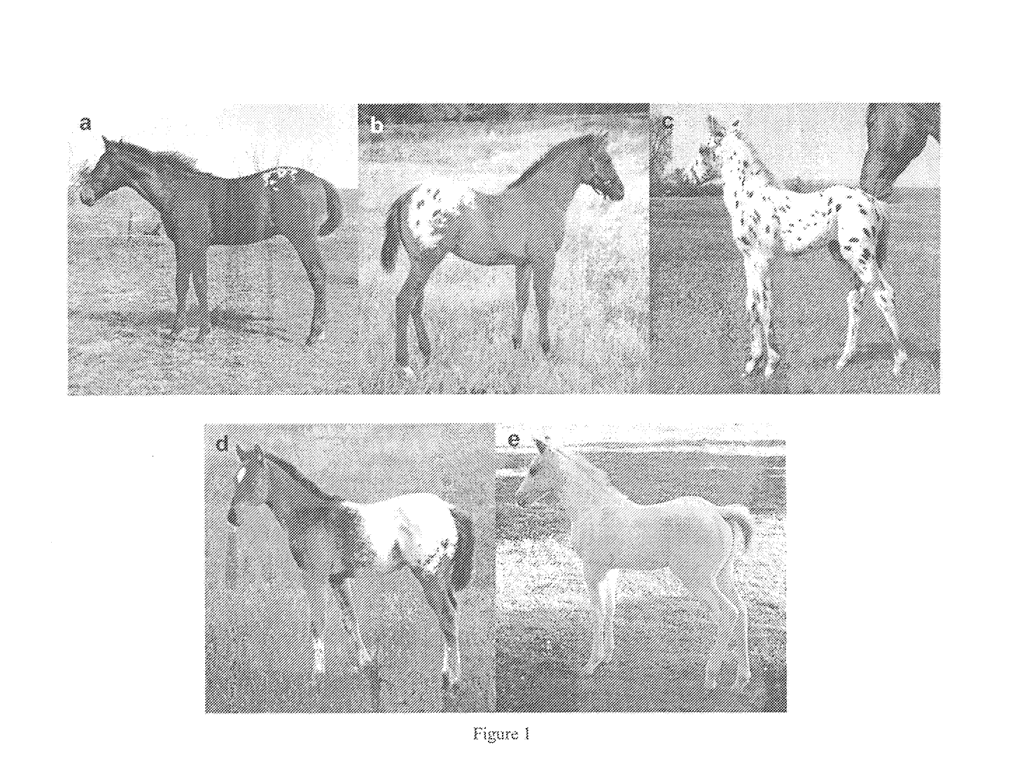

The phrase “different coat patterns” as used herein refers to variations in coat color, coat spotting, and coat patterns. Embodiments of different coat patterns are shown in FIG. 1.

The phrase “detecting or selecting different coat patterns” refers to a method or process of determining if a horse has or does not have or will have or will not have a specific coat pattern, and includes determining the type of coat pattern.

The phrase “determining the presence of at least one SNP allele associated with LP” as used herein includes, without limitation, obtaining a sample and testing the sample for hybridization with a probe that hybridizes to the SNP allele or obtaining a sample, amplifying from the sample a region surrounding the SNP allele and analyzing the amplified region for the nucleotide at the SNP position. Analysis of the amplified region for the nucleotide at the SNP position includes, without limitation, sequencing the region, or subjecting the amplified region to any SNP genotyping assay such as a restriction fragment length polymorphism among others.

One SNP genotyping assay is a Custom TaqMan® SNP Genotyping Assay which involves two primers and two probes. Primers correspond to the region of DNA amplified during a polymerase chain reaction. Each probe is completely complementary to only one allele and each probe is labeled with a different reporter fluorophore. Therefore alleles are determined by fluorescence detected (see Table 2 as an example).

In one embodiment, the method disclosed herein can be used to identify the carriers of the recessive lp factor (LP/lp). As explained previously, a single autosomal dominant gene, Leopard Complex (LP), is responsible for inheritance of these coat patterns. Horses homozygous for leopard complex spotting (LP/LP) have fewer spots on the white patterned areas than heterozygotes (LP/lp). Inheritance of the leopard complex spotting in horses can be in three forms: (1) LP/LP=few to no spots of pigment; (2) LP/lp=carrier (plentiful spots of pigment in white areas of coat); (3) lp/lp=true solid coat.

Accordingly, in one embodiment, the methods for detecting or selecting coat patterns in a horse further comprise selecting a horse that has no allele associated with LP and selecting a horse that has two copies of an allele associated with LP, breeding the selected horses together to generate a horse with an LP/lp genotype. In yet another embodiment, the methods further comprise selecting two horses, each with two copies of an allele associated with LP and breeding the selected horses together to generate a horse with an LP/LP genotype. In yet a further embodiment, the methods further comprise selecting two horses, each with no alleles associated with LP and breeding the selected horses together to generate a horse with an lp/lp genotype or selecting a horse with no alleles associated with LP and breeding the selected horse with a Thoroughbred, quarter horse, or other lp/lp horse to generate a horse with an lp/lp genotype. In yet another embodiment, the methods further comprise selecting two horses, each with one copy of an allele associated with LP and breeding the horses to generate a horse with an LP/LP, LP/lp or lp/lp genotype. In yet another embodiment, the methods further comprise selecting two horses, the first with one allele associated with LP and the second with two alleles associated with LP and breeding the selected horses to generate a horse with an LP/LP or LP/lp genotype. In yet a further embodiment, the methods further comprise selecting two horses, the first with one allele associated with LP and the second with no alleles associated with LP and breeding the selected horses to generate a horse with an LP/lp or lp/lp genotype. In one embodiment, the horse is an Appaloosa. In another embodiment, the horse is a Knabstrupper. In yet another embodiment, the horse is an American Miniature. In a further embodiment, the horse is a British Spotted Pony. In yet a further embodiment, the horse is a Pony of the Americas.

It is contemplated that the methods described herein can be used in combination with other methods to select or detect horse coat patterns, including genotyping and/or phenotypic observations. As one example, the methods described herein can be used in combination with genetic testing of horse coat colour offered by many laboratories, including without limitation, by UC Davis Veterinary Genetics Lab; Genetic Technologies Corp., Victoria, Australia; Animal Genetics in Florida and the United Kingdom and/or VetGen in Ann Arbor, Mich. In another embodiment, the method can be used in combination with determining the LP genotype of the horse.

### (iii) Agents to Detect Single Nucleotide Polymorphisms

The determination of a single nucleotide polymorphism disclosed herein is optionally carried out by detecting binding of a nucleic acid sequence that specifically hybridizes to the associated allele. Accordingly, in one embodiment, the present application provides a probe that specifically hybridizes to at least one of the SNP alleles associated with CSNB or LP. In one embodiment, the single nucleotide polymorphism is located at position 108281765 of ECA1 (horse chromosome 1) and the allele associated with CSNB or LP is C. In another embodiment, the single nucleotide polymorphism is located at position 108288853 of ECA1 (horse chromosome 1) and the allele associated with CSNB or LP is T. In yet another embodiment, the single nucleotide polymorphism is located at position 108337089 of ECA1 (horse chromosome 1) and the allele associated with CSNB or LP is G.

The term “a probe that specifically hybridizes to the associated allele” as used herein refers to a nucleic acid that binds to a sequence comprising the SNP associated allele and not to a sequence having a different nucleotide at the SNP position. For example, for the SNP at position 108281765 of ECA1, the probe that specifically hybridizes to the associated allele would be a probe that binds a sequence that contains the C nucleotide but not a sequence that contains a different allele at that same position, such as a T nucleotide (unless of course one was investigating the complementary strand as described above). In one embodiment, the probe comprises the nucleotide sequence as shown in SEQ ID NO:14. For the SNP at position 1082888853 of ECA1, the probe that specifically hybridizes to the associated allele would be a probe that binds a sequence that contains the T nucleotide but not a sequence that contains a different allele at that same position, such as a C nucleotide. In one embodiment, the probe comprises the nucleotide sequence as shown in SEQ ID NO:13. For the SNP at position 108337089 of ECA1, the probe that specifically hybridizes to the associated allele would be a probe that binds a sequence that contains the G nucleotide but not a sequence that contains a different allele at that same position, such as a T nucleotide. In one embodiment, the probe comprises the nucleotide sequence as shown in SEQ ID NO:15.

The term “probe” as used herein refers to an isolated nucleic acid molecule having a nucleic acid sequence that will hybridize to a nucleic acid target sequence. In one example, the probe hybridizes to a TRPM1 DNA or a nucleic acid sequence complementary to the TRPM1 DNA. The length of probe depends on the hybridization conditions and the sequences of the probe and nucleic acid target sequence. In one embodiment, the probe is at least 8, 10, 15, 20, 25, 50, 75, 100, 150, 200, 250, 400, 500 or more nucleotides in length.

The term “isolated nucleic acid sequence” as used herein refers to a nucleic acid substantially free of cellular material or culture medium when produced by recombinant DNA techniques, or chemical precursors, or other chemicals when chemically synthesized. An “isolated nucleic acid” is also substantially free of sequences which naturally flank the nucleic acid (i.e. sequences located at the 5′ and 3′ ends of the nucleic acid) from which the nucleic acid is derived. The term “nucleic acid” is intended to include DNA and RNA and can be either double stranded or single stranded. The nucleic acid sequences contemplated by the present disclosure include isolated nucleotide sequences which hybridize to a RNA product or DNA of the present disclosure, nucleotide sequences which are complementary to the RNA product or DNA of the disclosure, nucleotide sequences which act as probes, or nucleotide sequences which are sets of specific primers.

The term “hybridize” refers to the sequence specific non-covalent binding interaction with a complementary nucleic acid. In one embodiment, the hybridization is under stringent hybridization conditions. In another embodiment, the hybridization is under moderately stringent conditions.

By “hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrid, is determined by the Tm, which in sodium containing buffers is a function of the sodium ion concentration and temperature (Tm=81.5° C.−16.6 (Log 10 [Na+])+0.41(% (G+C)−600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm. For example if nucleic acid molecules are sought that have a >95% identity, the final wash temperature will be reduced by about 5° C. Based on these considerations those skilled in the art will be able to readily select appropriate hybridization conditions. In some embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm—5° C. based on the above equation, followed by a wash of 0.2×SSC/0.1% SDS at 60° C. Moderately stringent hybridization conditions include a washing step in 3×SSC at 42° C. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 1989, 6.3.1-6.3.6 and in: Sambrook et al., Molecular Cloning, a Laboratory Manual, Cold Spring Harbor Laboratory Press, 1989, Vol. 3.

In another embodiment, the detection of a single nucleotide polymorphism disclosed herein is optionally determined by analyzing the region comprising the SNP, for example, by sequencing the region comprising the SNP or by subjecting an amplified region to any SNP genotyping assay such as a restriction fragment length polymorphism or a Custom TaqMan®SNP Genotyping Assay as described herein. In one embodiment, two or more isolated nucleic acid sequences that are specific primers are able to amplify the sequence containing the SNP. Pairs of primers may be selected wherein one primer is upstream of the SNP location and one primer is downstream of the SNP location.

The term “primer” as used herein refers to a nucleic acid sequence, whether occurring naturally as in a purified restriction digest or produced synthetically, which is capable of acting as a point of synthesis when placed under conditions in which synthesis of a primer extension product, which is complementary to a nucleic acid strand, is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less. The factors involved in determining the appropriate length of primer are readily known to one of ordinary skill in the art.

For the SNP found at position 108281765 of ECA1, one primer is upstream of the nucleotide at position 108281765 and another primer is downstream from the nucleotide at position 108281765. In one embodiment, a forward primer having the nucleotide sequence 5′-ACTGGGGAGTCTGTCCACTG-3′ (SEQ ID NO:1) and a reverse primer having the nucleotide sequence 5′-CTTAGCTCAAGCCCCTTCCT-3′ (SEQ ID NO:2) amplify the region surrounding the SNP found at position 108281765. In another embodiment, a forward primer having the nucleotide sequence 5′-CAGCAGGGATAACACTTGTACAGT-3′ (SEQ ID NO:9) and a reverse primer having the nucleotide sequence 5′-TGATAAGAATAATGACCAGGACCTCCAT-3′ (SEQ ID NO:10) amplify the region surrounding the SNP found at position 108281765. The amplified sequence is then sequenced or otherwise analyzed to determine the identity of the nucleotide at position 108281765.

For the SNP found at position 108288853 of ECA1, one primer is upstream of the nucleotide at position 108288853 and another primer is downstream from the nucleotide at position 108288853. In one embodiment, a forward primer having the nucleotide sequence 5′-CAGTGAACCGGGCTCTTAAA-3′ (SEQ ID NO:3) and a reverse primer having the nucleotide sequence 5′-GCAGCCTCCAAAACTGACTC-3′ (SEQ ID NO:4) amplify the region surrounding the SNP found at position 108288853. In another embodiment, a forward primer having the nucleotide sequence 5′-GCTTCGTGACGCTCTGCTT-3′ (SEQ ID NO:7) and a reverse primer having the nucleotide sequence 5′-CCCCAAATCGTGAACTTGCATT-3′ (SEQ ID NO:8) amplify the region surrounding the SNP found at position 108288853. The amplified sequence is then sequenced or otherwise analyzed to determine the identity of the nucleotide at position 108288853.

For the SNP found at position 108337089 of ECA1, one primer is upstream of the nucleotide at position 108337089 and another primer is downstream from the nucleotide at position 108337089. In one embodiment, a forward primer having the nucleotide sequence 5′-CTTAGGTGAGGCGAGGTCAG-3′ (SEQ ID NO:5) and a reverse primer having the nucleotide sequence 5′-ACCTGGCAGGCCTATCTTCT-3′ (SEQ ID NO:6) amplify the region surrounding the SNP found at position 108337089. In another embodiment, a forward primer having the nucleotide sequence 5′-TGGGTACATGTCACCTGCAATG-3′ (SEQ ID NO:11) and a reverse primer having the nucleotide sequence 5′-TTGATCACCATGGGAATACATTGCT-3′ (SEQ ID NO:12) amplify the region surrounding the SNP found at position 108337089. The amplified sequence is then sequenced or otherwise analyzed to determine the identity of the nucleotide at position 108337089.

### (C) Kits

Another aspect of the present disclosure is a kit for screening, detecting, or diagnosing congenital stationary night blindness in a subject or detecting or screening horse coat patterns. In one embodiment, the kit comprises a probe that specifically hybridizes to a SNP allele as disclosed herein or specific primers that amplify a region comprising a SNP allele as disclosed herein and/or instructions for use. The kit can also include ancillary agents. For example, the kits can include vessels for storing or transporting the probes and/or primers; a control; instruments for obtaining a sample; and/or buffers or stabilizers. The kits can also include sequencing reagents or restriction enzymes for restriction fragment length polymorphism analysis. In another embodiment, the kit comprises a probe that specifically hybridizes to a SNP allele and a probe that specifically hybridizes to the wild-type allele, wherein the probes are distinctly labeled, for example, using different fluorophores, such as fluorophore 6-carboxyfluoroscein (FAM) and fluorophore 4,7,2′-trichloro-7′-phenyl-6-carboxyfluorescein (VIC). In one embodiment, the probes comprise SEQ ID NO:13 and SEQ ID NO:16. In another embodiment, the probes comprise SEQ ID NO:14 and SEQ ID NO:17. In yet another embodiment, the probes comprise SEQ ID NO:15 and SEQ ID NO:18.

The above disclosure generally describes the present application. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

### Genotyping:

DNA was isolated from blood or hair of unrelated horses from breeds segregating for LP: Appaloosa (N=205), Knabstrupper (N=66), Noriker (N=112), American Miniature (N=63), Pony of the America (N=20), British Spotted Pony (N=25), and Australian Spotted Pony (N=10). DNA was also isolated from the Thoroughbred (N=37) and American Quarter Horse (N=3), which are breeds not segregating for LP. LP genotype and CSNB status were determined as previously described (Bellone et al. 2008). Initially, six SNPs were investigated by PCR amplification and sequencing of DNA from 10 individuals (Table 1). The primers, annealing temperatures and PCR product sizes of the SNPs tested are described in Table 1. Three of these SNPs (ECA1 g.108489901G>A, ECA1 g.108497669C>A, and ECA1 g.108497990C>T) did not show complete association with LP genotype in this panel and were excluded as the causative mutation and thus not investigated further. The other three SNPs (ECA1 g.108281765T>C, ECA1 g.108288853C>T, and ECA1 g.108337089T>G) showed complete association and were either genotyped by direct sequencing or by custom TaqMan genotyping assays in 531 additional individuals (Assay IDs AHS0Q19, AHRRSV1, and AHT9O8H, Applied Biosystems) (Table 2). All TaqMan assays were performed in 5-μL reactions on a Mastercyler® ep realplex thermocycler (Eppendorf).

SNP Association with LP and CSNB:

ECA1 g.108281765T>C, ECA1 g.108288853C>T, and ECA1 g.108337089T>G genotypes were analyzed for association with LP genotype and CSNB status by chi-squared analysis. All three SNPs were completely associated with LP (N=513, X²=1026, P<0.0005) and CSNB (N=28, X²=28, P<0.0005). It is possible that one of these SNPs is the causative mutation for LP and CSNB. However, none of these are located in exonic regions that have previously been characterized, and thus further investigation is warranted (Bellone et al. 2010). It is also likely that these SNPs may simply be tightly associated and are not causative. Nevertheless, any or all of these SNPs could be used as a DNA test for LP and CSNB.

### Example 2

### Congenital Stationary Night Blindness is Associated with the Leopard Complex in the Miniature Horse

The objective of this Example was to determine if congenital stationary night blindness (CSNB) exists in the miniature horse in association with leopard complex spotting patterns (LP), and to investigate if CSNB in the miniature horse is associated with three single nucleotide polymorphisms (SNPs) in the region of TRPM1 that are highly associated with CSNB and LP in Appaloosas.

Three groups of miniature horses were studied based on coat patterns suggestive of LP/LP (n=3), LP/lp (n=4), and lp/lp genotype (n=4). Horses were categorized based on phenotype as well as pedigree analysis as LP/LP, LP/lp, and lp/lp. Neurophthalmic examination, slit-lamp biomicroscopy, indirect ophthalmoscopy, and scotopic flash electroretinography were performed on all horses. Hair samples were processed for DNA analysis. Three SNPs identified and associated with LP and CSNB in the Appaloosa were investigated for association with LP and CSNB in these Miniature horses.

### Results

### Clinical Examination

Abnormalities noted on slit-lamp biomicroscopy included iris to iris persistent pupillary membranes in all horses (11 of 11), incipient posterior cortical cataract (one of three LP/LP), incipient nuclear and equatorial cataract (one of four LP/lp) and immature cataract (one of three LP/LP). Strabismus was not noted in any horse. Indirect ophthalmoscopy showed no abnormalities in any horse.

### Electroretinography

Dark-adapted flash ERG recordings consisted of a negative a-wave and absence of a b-wave consistent with CSNB in all LP/LP (N=3) horses. Both a- and b-waves were present in all LP/lp and lp/lp horses.

### SNP Analysis

ECA1 g.108281765T>C, ECA1 g.108288853C>T, and ECA1 g.108337089T>G were completely associated with predicted LP genotype (X²=22, P<<0.0005) and CSNB status (X²=11, P<0.0005) (Table 3). All CSNB affected Miniature horses were homozygous for the alleles previously associated with LP and CSNB in the Appaloosa, where unaffected horses were either heterozygous for the SNPs or did not have the associated alleles. Thus confirming that the in the Miniature Horse, CSNB is perfectly associated with LP and SNPs in the candidate gene TRPM1.

### Materials and Methods

### Animals

Eleven breeder-owned Miniature horses ranging in age from 1 to 13 years of age were studied. Three groups of animals were formed based on phenotype suggestive of LP/LP (n=3, two male/one female), LP/lp (n=4, three male/one female), and lp/lp (n=4, all female). Phenotyping was performed as previously described (Bellone et al. 2008). All animals were unrelated by at least one degree of separation. The Canadian Council on Animal Care guidelines for experimental animal use was followed, and the University of Saskatchewan Animal Care Committee approved the research protocol.

### Clinical Examination

Ophthalmic examination completed by a veterinary ophthalmologist included neurophthalmic examination, slit-lamp biomicroscopy (SL-14; Kowa, Tokyo, Japan), and indirect ophthalmoscopy (Heine Omega 200; Heine Instruments, Kitchener, ON, Canada). Horses were sedated with 0.4 mg/kg intravenous xylazine (Rompun; Bayer, Inc. Toronto, ON, Canada). Pharmacological mydriasis was achieved with 0.2 mL 1% tropicamide (1% Mydriacyl; Alcon, Canada, Mississauga, ON, Canada). Auriculopalpebral nerve blocks were performed using 2 mL of a 2% lidocaine hydrochloride injectable solution (Bimeda-MTC Animal Health Inc., Cambridge, ON, Canada).

### Electroretinography

Scotopic flash ERGs were completed bilaterally. A corneal DTL microfiber electrode (DTL Plus Electrode; Diagnosys LLC, Littleton, Mass.) was placed on the cornea and attached to the skin with glue (Krazy Glue; Elmer Products Canada Corp, Toronto, ON, Canada) at the medial and lateral canthi. Platinum subdermal needle electrodes (Cadwell Low Profile Needle electrodes; Cadwell Laboratories, Kenewick, Wash., USA) were used as reference and ground. The reference electrode was placed subdermally 3 cm from the lateral canthus and the ground electrode was placed subdermally over the occipital bone. The ERGs were elicited with a white xenon strobe light. Eyelids were held open manually for each test. ERGs were recorded with a Cadwell Sierra II (Cadwell Laboratories). Horses were dark adapted for 20 min prior to recording an average of 10 responses to a bright white flash.

### DNA Analysis

DNA was isolated from all animals from mane or tail hair follicles as previously described. (Locke et al. 2002) Three SNPs associated with LP and CSNB in the Appaloosa (ECA1 g.108281765T>C, ECA1 g.108288853C>T, and ECA1 g.108337089T>G were genotyped by custom TaqMan genotypingassays (Assay IDs AHS0Q19, AHRRSV1, and AHT9O8H; Applied Biosystems, Foster City, Calif., USA) (Table 2; Bellone et al. 2010). Reactions were performed in a total volume of 10 IL on a StepOne-Plus™ real-time PCR system (Applied Biosystems). SNPs were analyzed for association with LP genotype (identified based on coat pattern phenotype) and CSNB status (identified based on ERG results) by chi-square analysis.

### Discussion

Miniature horses are popular in Europe and the Americas as companion animals, show horses, and as service animals, similar to assistance dogs for people with disabilities. They are friendly, easy to handle, and interact well with people. The designation of miniature horse is determined by height of the animal. This varies depending on the particular breed registry but is usually less than 34-38 inches at the withers (http://www.amha.org, http://www.britishspottedponysociety.co.uk, http://www.knabstrupperforeningen.dk/sider/englishindex.htm, http://www.falabellafmha.com). Miniature horses were developed from multiple different pony and full-size breeds. Specifically, appaloosa-spotted miniature horses are descended from three main ancestral sources: appaloosa spotted Spanish Horses, the Knabstrupper breed of Denmark, as well as the British Spotted Pony of England (http://www.amha.org, http://www.britishspottedponysociety.co.uk, http://www.knabstrupperforeningen.dk/sider/englishknabstruphistory.htm, http://www.falabellafmha.com).

The main finding of this Example is that the Miniature horse breed is affected by CSNB in association with homozygosity of the LP allele as in the Appaloosa. This is based on ERG results as well as DNA testing. The present inventors have previously determined that a defect in expression of the TRPM1 gene is likely responsible for both LP and CSNB as TRPM1 expression (as measured by qRT-PCR) is dramatically reduced in the retina and skin of CSNB (LP/LP) Appaloosas (Bellone et al. 2008). TRPM1 mutations have recently been confirmed in autosomal recessiveforms of SB-CSNB in humans by several independent groups (Audo et al. 2009; van Genderen et al. 2009; Li et al. 2009; Nakamura et al. 2009).

As discussed herein, TRPM1 belongs to a family of transient receptor potential (TRP) channels. TRPs are important in cellular and somatosensory perception (Nilius 2007). TRPM1 is also known as melastatin and is downregulated in highly metastatic melanoma cells, suggesting that this protein plays an important role in normal melanogenesis (Duncan et al. 2001). Furthermore it has been recently shown that TRPM1 expression is correlated with melanin content in neonatal epidermal melanocytes suggesting a role in pigment storage (Oancea et al. 2009). Additionally, TRPM1 is now known to be an important channel responsible for normal retinal function (van Genderen et al. 2009; Morgans et al. 2009; Koike et al. 2010a; Koike et al. 2010b).

Retinal function is measured using ERG recordings. The a-wave of the ERG represents the photoreceptor response to light stimulation, while the b-wave directly relates to bipolar cell activity (Kofuji et al. 2000). The dark-adapted ERG recordings in the Miniature horse in this Example were characteristic and similar to those reported previously in CSNB-affected horses (Witzel et al. 1977a; Witzel et al. 1977b; Witzel et al. 1978; Rebhun et al. 1984; Sandmeyer et al. 2007; Nunnery et al. 2005). In this study, however, the main purpose was to confirm a diagnosis of CSNB-affected Miniature horses rather than study the ERG characteristics with more in-depth protocols. The lack of a dark-adapted b-wave in the ERG of horses with CSNB localizes the physiologic abnormality to a defect in neural transmission of the rod bipolar cells, which in the rod system, are all of the ON-center type.

In a normally functioning retina light stimulation results in closure of sodium channels in the photoreceptor cell membrane leading to hyperpolarization (a-wave). Hyperpolarization of the photoreceptor diminishes the release of glutamate (Nawy 2000). An ON bipolar cell will react to this change by depolarizing (b-wave). Depolarization of the ON-bipolar cell is controlled by metabotropic glutamate receptor 6 (mGluR6). mGluR6 is coupled to a heterotrimeric G-protein complex Go. When activated, the alpha subunit, Gαo, ultimately closes a downstream cation channel (Weng et al. 1997; Vardi et al. 1998). The decrease in glutamate that occurs with photoreceptor hyperpolarization inactivates the mGluR6 channel leading to an open state of the cation channel and thus depolarization (Koike et al. 2010a). The molecular identity of the cation channel that generates the depolarizing current is now known to be TRPM1 (Koike et al. 2010a; Koike et al. 2010b). In CSNB the absence of TRPM1 prevents depolarization of the ON bipolar cell which corresponds to an absent b-wave in the ERG.

Proof that TRPM1 is the cation channel responsible for the b-wave of the ERG was obtained through patch-clamp recording techniques (Morgans et al. 2009; Koike et al. 2010a; Koike et al. 2010b). Immunohistologic data show that TRPM1 localizes in human retina specifically to the ON bipolar cell dendrites (Koike et al. 2010a; Koike et al. 2010b). Additionally, TRPM1 mutant mice (TRPM1−/−) show similar ERG characteristics as CSNB-affected horses and humans (Morgans et al. 2009; Koike et al. 2010a; Koike et al. 2010b). However, TRPM1 mutant mice and humans with TRPM1 mutations have not been reported to have spotting patterns or pigmentation differences. Thus, the role of TRPM1 in regulation of LP coat patterns is of yet undetermined. It has, however, been suggested that TRPM1 ion channel function is critical for normal melanocyte pigmentation and thus human pigmentation disorders caused by TRPM1 and its role in LP pigmentation maybe identified in the future (Oancea et al. 2009).

Previous work allowed for fine-mapping of the mutations of the LP and CSNB locus to a 173-kb haplotype on ECA1 involving TRPM1. Targeted resequencing this region identified six SNPs for further investigation, three (ECA1 g. 108281765T>C, ECA1 g.108288853C>T, ECA1 g.108337089T>G) of which were completely associated with LP and CSNB (Example 1). In this Example, it was confirmed that these three SNPs were also completely associated with LP and CSNB in another breed, the Miniature horse.

It is likely that CSNB is also completely associated with LP in other horse breeds where LP spotting is present. Although CSNB is described in a Thoroughbred and a Paso Fino, LP is not thought to be present in these breeds, thus, CSNB in these breeds is likely caused by a different genetic mechanism, similar to what is seen in humans where CSNB has been reported to be caused by mutations in TRPM1, as well as NYX, GRM6, and SLC24A1 (Nakamura et al. 2010; Bech-Hansen et al. 2000; Zeitz et al. 2005; Riazuddin et al. 2010).

At the moment, electroretinography is the only method of confirming that a horse has CSNB. Using coat pattern alone to predict LP genotype, and thus, CSNB has limitations. LP patterning can develop with age. Even though some evidence is usually present at birth, this is not always the case (Witzel et al. 1977b). A horse's status with respect to LP can best be determined by visual inspection when there is enough dense white patterning present at birth to distinguish between heterozygous (spotted) and homozygous (fewspotted) horses. Horses with little or no white patterning at birth cannot be classified by phenotype. Additionally horses with gray in their background will lose pigment with age making it difficult to distinguish the leopard complex patterns (Witzel et al. 1977b). Furthermore, Leopard complex horses with additional white spotting phenotypes (sabino, tobiano, splash, and overo) are often difficult to classify by phenotype alone as these can incorrectly be classified as homozygous (termed ‘false few-spot’ or ‘false snowcap’). In the present study, horses with gray in the background or additional white spotting phenotypes were excluded from the study to avoid incorrect phenotypic classification. The three SNPs identified have not been shown to be the causative mutation of CSNB or LP, however, their complete association suggests genotyping of these SNPs is useful as a genetic test for both.

While the present disclosure has been described with reference to what are presently considered to be the examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Candidate SNPs investigated for LP and CSNB. PCR conditions
and results of SNP association with LP genotype in
10 animals from five different breeds are presented:

| | | | | PCR | Association |
|---|---|---|---|---|---|
| | | | Annealing | Product | with LP |
| | Foward primer | Reverse primer | Temp. | Size | genotype in |
| SNP Tested | sequence | sequence | (° C.) | (Bp) | small panel |
| ECA1 | 5′- | 5′- | 65 | 326 | 10/10 |
| g.108281765T>C | ACTGGGGAGTCTGTCCACTG- | CTTAGCTCAAGCCCCTTCCT- | | | associated* |
| | 3′ (SEQ ID NO: 1) | 3′ (SEQ ID NO: 2) | | | |
| ECA1 | 5′- | 5′- | 58 | 223 | 10/10 |
| g.108288853C>T | CAGTGAACCGGGCTCTTAAA- | GCAGCCTCCAAAACTGACTC- | | | associated* |
| | 3′ (SEQ ID NO: 3) | 3′ (SEQ ID NO: 4) | | | |
| ECA1 | 5′- | 5′- | 65 | 485 | 10/10 |
| g.108337089T>G | CTTAGGTGAGGCGAGGTCAG- | ACCTGGCAGGCCTATCTTCT- | | | associated* |
| | 3′ (SEQ ID NO: 5) | 3′ (SEQ ID NO: 6) | | | |
| ECA1 | 5′- | 5′- | 65 | 153 | 8/10 |
| g.108489901G>A | TGTCCTCGGCCACGCCC- | GTCAGCCCCCAGCCACAT- | | | associated |
| | 3′ (SEQ ID NO: 19) | 3′ (SEQ ID NO: 20) | | | |
| ECA1 | 5′- | 5′- | 60 | 246 | 9/10 |
| g.108497669C>A | CCTGCACCGTGACTTCTATG- | GCCAAGCTGGAGAGAACACT- | | | associated |
| | 3′ (SEQ ID NO: 21) | 3′ (SEQ ID NO: 22) | | | |
| ECA1 | 5′- | 5′- | 62 | 306 | 8/10 |
| g.108497990C>T | TTCTCTCCAGCTTGGCAGTT- | GATGCTCAGTTCAGCCACAA- | | | associated |
| | 3′ (SEQ ID NO: 23) | 3′ (SEQ ID NO: 24) | | | |

*SNPs with complete association were investigated further and were completely associated in a panel of 541 horses.
CSNB—congenital stationary blindness;
LP—leopard complex spotting

TABLE 2
PCR Primers and probes to detect LP and CSNB associated SNPs

| Forward | | Reverse | | | | Associate | Associated |
|---|---|---|---|---|---|---|---|
| Primer | Forward | Primer | Reverse | Probe 1 | Probe 1 | dSNP Probe | SNP Probe |
| Name | Primer Seq. | Name | Primer Seq. | Name | Sequence | Name | Sequence |
| SNP8853_F | GCTTCGTGACG | SNP8853_R | CCCCAAATCGT | SNP8853_V | ACGTCTGG | SNP8853_M | ACGTCTGGGT |
| | CTCTGCTT | | GAACTTGCATT | | GTTGGAAG | | TAGAAG(SEQ |
| | (SEQ ID NO: | | (SEQ ID NO: | | (SEQ ID | | ID NO: 13) |
| | 7) | | 8) | | NO: 16) | | |
| SNP1765_F | CAGCAGGGATA | SNP1765_R | TGATAAGAATA | SNP1765_V | TGCAAGGT | SNP1765_M | CAAGGTACCG |
| | ACACTTGTACA | | ATGACCAGGAC | | ACTGGATT | | GATTTA(SEQ |
| | GT (SEQ ID | | CTCCAT (SEQ | | TA (SEQ | | ID NO: 14) |
| | NO: 9) | | ID NO:10) | | ID NO: | | |
| | | | | | 17) | | |
| SNP7089_F | TGGGTACATGT | SNP7089_R | TTGATCACCAT | SNP7089_V | CTCGAGAC | SNP7089_M | CTCGAGACTC |
| | CACCTGCAATG | | GGGAATACATT | | TCAATCTG | | ACTCTGTT |
| | (SEQ ID NO: | | GCT (SEQ ID | | TT (SEQ | | (SEQ ID |
| | 11) | | NO: 12) | | ID NO: | | NO: 15) |
| | | | | | 18) | | |

Probe 1 Dye: VIC;
Probe 2 (Associated SNP) Dye: FAM

TABLE 3
Association of leopard complex spotting patterns (LP), congenital
stationary night blindness (CSNB) and single nucleotide polymorphisms
(SNPs) in TRPM1 in the Miniature Horse

| SNP ECA1 g.108281765 T > C | | | |
|---|---|---|---|
| LP genotype and CSNB status | C/C | T/C | T/T |
| Affected (LP/LP) | 3 | 0 | 0 |
| Unaffected (LP/lp) | 0 | 4 | 0 |
| Unaffected (lp/lp) | 0 | 0 | 4 |
| SNP ECA1 g.108288853 C > T | | | |
| LP genotype and CSNB status | T/T | T/C | C/C |
| Affected (LP/LP) | 3 | 0 | 0 |
| Unaffected (LP/lp) | 0 | 4 | 0 |
| Unaffected (lp/lp) | 0 | 0 | 4 |
| SNP ECA1 g.108337089 T > G | | | |
| LP genotype and CSNB status | G/G | G/T | T/T |
| Affected (LP/LP) | 3 | 0 | 0 |
| Unaffected (LP/lp) | 0 | 4 | 0 |
| Unaffected (lp/lp) | 0 | 0 | 4 |

All three SNPs tested were completely associated with LP and CSNB (N = 11).
CSNB, congenital stationary night blindness; SNP, single nucleotide polymorphism.

### REFERENCES

Audo I, Kohl S, Leroy B P et al. TRPM1 is mutated in patients with autosomal recessive complete congenital stationary night blindness. American Journal of Human Genetics 2009; 85:720-729.
Bech-Hansen N T, Naylor M J, Maybaum T A et al. Mutations in NYX, encoding the leucine-rich proteoglycan nyctalopin, cause X-linked complete congenital stationary night blindness. Natural Genetics 2000; 26: 319-323.
Bellone, R., T. Lear, D. L. Adelson and E. Bailey, 2006a Comparative mapping of oculocutaneous albinism type II (OCA2), transient receptor potential cation channel, subfamilyMmember 1 (TRPM1) and two equine microsatellites, ASB08 and 1CA43, among four equid species by fluorescence in situ hybridization. Cytogenet. Genome Res. 114: 93A.
Bellone R R, Brooks S A, Sandmeyer L, et al. Differential gene expression of TRPM1, the potential cause of congenital stationary night blindness and coat spotting patterns (LP) in the Appaloosa horse (Equus caballus). Genetics. 2008; 179:1861□1870.
Bellone, R., S. Lawson, N. Hunter, S. Archer and E. Bailey, 2006b Analysis of a SNP in exon 7 of equine OCA2 and its exclusion as a cause for appaloosa spotting. Anim. Genet. 37: 525.
Bennett, D. C., and M. L. Lamoreux, 2003 The color loci of mice: a genetic century. Pigment Cell Res. 16: 333-344.
Berger, S. A., 2006 Signaling pathways influencing SLF and c-kit mediated survival and proliferation. Immunol. Res. 35: 1-12.
Brooks, S. A., and E. Bailey, 2005 Exon skipping in the KIT gene causes a Sabino spotting pattern in horses. Mamm. Genome 11: 893-899.
Brooks, S., T. L. Lear, D. Adelson and E. Bailey, 2007 A chromosome inversion near the KIT gene and the Tobiano spotting pattern in horses. Cytogenet. Genome Res. 119: 225-230.
Brunberg, E., L. Andersson, G. Cothran, K. Sandberg, S. Mikko et al., 2006 A missense mutation in PMEL17 is associated with the silver coat color in the horse. BMC Genet. 7: 46.
Clapham, D. E., L. W. Runnels and C. Strübing, 2001 The TRP ion channel family. Nat. Rev. Neurosci. 2: 387-396.
Deeds, J., F. Cronin and L. M. Duncan, 2000 Patterns of melastatin mRNA expression in melanocytic tumors. Hum. Pathol. 31: 1346-1356.
Duffy, D. L., G. W. Montgomery, W. Chen, Z. Z. Zhao, L. Le et al., 2007 A three-single-nucleotide polymorphism haplotype in intron 1 of OCA2 explains most human eye-color variation. Am. J. Hum. Genet. 80: 241-252.
Duncan, L. M., J. Deeds, J. Hunter, J. Shao, L. M. Homgren et al., 1998 Down-regulation of the novel gene melanstatin correlates with potential for melanomametastasis. Cancer Res. 58:1515-1520.
Duncan, L. M., J. Deeds, F. E. Cronin, M. Donovan, A. J. Sober et al., 2001 Melastatin expression and prognosis in cutaneous malignant melanoma. J. Clin. Oncol. 19: 568-576.
Eiberg, H., J. Troelsen, M. Nielsen, A. Mikkelsen, J. Mengel-From et al., 2008 Blue eye color in humans may be caused by a perfectly associated founder mutation in a regulatory element located in the HERC2 gene inhibiting OCA2 expression. Hum. Genet. 123: 177-187.
Erickson, C. A., 1993 From the crest to the periphery: control of pigment cell migration and lineage segregation. Pigment Cell Res. 6: 336-347.
Fang, D., and V. Setaluri, 2000 Expression and up-regulation of alternatively spliced transcripts of melastatin, a melanoma metastasis-related gene, in human melanoma cells. Biochem. Biophys. Res. Commun. 279: 53-61.
Gardner, J. M., Y. Nakatsu, Y. Gondo, S. Lee, M. F. Lyon et al., 1992 The mouse pink-eyed dilution gene: association with human Prader-Willi and Angelman syndromes. Science 257: 1121-1124.
Gommerman, J. L., and S. A. Berger, 1998 Protection from apoptosis by steel factor but not interleukin-3 is reversed through blockade of calcium influx. Blood 91: 1891-1900.
Haase, B., S. A. Brooks, A. Schlumbaum, P. Azor, E. Bailey et al., 2007 Allelic heterogeneity at the equine KIT locus in dominant white (W) horses. PLoS Genet. 3: e195.
Hunter, J. J., J. Shao, J. S. Smutko, B. J. Dussault, D. L. Nagle et al., 1998 Chromosomal localization and genomic characterization of the mouse melastatin gene (Mlsn1). Genomics 54: 116-123.
Kim, C., 2004 Transient receptor ion channels and animal sensation: lessons from Drosophila functional research. J. Biochem. Mol. Biol. 37: 114-121.
Kofuji P, Ceelen P, Zahs K R et al. Genetic inactivation of an inwardly rectifying potassium channel (Kir4.1 subunit) in mice: phenotypic impact in retina. Journal of Neuroscience 2000; 20:5733-5740.
Koike C, Numata T, Ueda H et al. TRPM1: a vertebrate TRP channel responsible for retinal ON bipolar cell function. Cell Calcium 2010a; 48: 95-101.
Koike C, Obara T, Uriu Y et al. TRPM1 is a component of the retinal ON-bipolar cell transduction channel in the mGluR6 cascade. Proceedings of the National Academy of Science USA 2010; 107:332-337.
Lapp, R. A., and G. Carr, 1998 Applied appaloosa color genetics. Appaloosa J. 52: 113-115.
Lee, S. T., R. D. Nicholls, R. E. Schnur, L. C. Guida, J. Lu-Kuo et al., 1994 Diverse mutations of the P gene among African-Americans with type II (tyrosinase-positive) oculocutaneous albinism (OCA2). Hum. Mol. Genet. 3: 2047-2051.
Li Z, Sergouniotis P I, Michaelides M et al. Recessive mutations of the gene TRPM1 abrogate ON bipolar cell function and cause complete congenital stationary night blindness in humans. American Journal of Human Genetics 2009; 85: 711-719.
Locke M M, Penedo M C, Bricker S J et al. Linkage of the grey coat colour locus to microsatellites on horse chromosome 25. Animal Genetics 2002; 33: 329-337.
Mariat, D., S. Taourit and G. Guerin, 2003 A mutation in the MATP gene causes the cream coat colour in the horse. Genet. Sel. Evol. 35: 119-133.
Marklund, L., M. J. Moller, K. Sandberg and L. Andersson, 1996 Amissensemutation in the gene for melanocyte-stimulating hormone receptor (MC1R) is associated with the chestnut coat color in horses. Mamm. Genome 7: 895-899.
Metallinos, D. L., A. T. Bowling and J. Rine, 1998 A missense mutation in the endothelin-B receptor gene is associated with Lethal White Foal Syndrome: an equine version of Hirschsprung disease. Mamm. Genome 9: 426-431.
Miller, R. W., 1965 Appaloosa coat color inheritance. Ph.D. Thesis, Animal Science Department, Montana State University, Bozeman, Mont.
Morgans C W, Zhang J, Jeffrey B G et al. TRPM1 is required for the depolarizing light response in retinal ON-bipolar cells. Proceedings of the National Academy of Science USA 2009; 106: 19174-19178.
Nakamura M, Sanuki R, Yasuma T R et al. TRPM1 mutations are associated with the complete form of congenital stationary night blindness. Molecular Vision 2010; 16: 425-437.
Nakanishi, S., Y. Nakajima, M. Masu, Y. Ueda, K. Nakahara et al., 1998 Glutamate receptors: brain function and signal transduction. Brain Res. Rev. 26: 230-235.
Nawy S. Regulation of the on bipolar cell mGluR6 pathway by Ca2+. Journal of Neuroscience 2000; 20: 4471-4479.
Ni-Komatsu, L., and S. J. Orlow, 2006 Heterologous expression of tyrosinase recapitulates the misprocessing and mistrafficking in oculocutaneous albinism type 2: effects of altering intracellular pH and pink-eyed dilution gene expression. Exp. Eye Res. 82: 519-528.
Nilius, B., 2007 TRP channels in disease. Biochim. Biophys. Acta 1772: 805-812.
Nomura, M., H. Iwakabe, Y. Tagawa, T. Miyoshi, Y. Yamashita et al., 1994 Developmentally regulated postsynaptic localization of a metabotropic glutamate receptor in rat rod biopolar cells. Cell 77: 361-369.
Nunnery C, Pickett P J, Zimmerman K L. Congenital stationary night blindness in a thoroughbred and a Paso Fino. Veterinary Ophthalmology 2005; 8: 415-419.
Oancea E, Vriens J, Brauchi S et al. TRPM1 forms ion channels associated with melanin content in melanocytes. Science Signaling 2009; 2: 1-11 http://www.sciencesignaling.org.
Ray, K., M. Chaki and M. Sengupta, 2007 Tyrosinase and ocular diseases: some novel thoughts on the molecular basis of oculocutaneous albinism type 1. Prog. Retin. Eye Res. 26: 323-358.
Rebhun, W. C., E. R. Loew, R. C. Riis and L. J. Laratta, 1984 Clinical manifestations of night blindness in the Appaloosa horse. Comp. Contin. Edu. Pract. Vet. 6: S103-S106.
Riazuddin S A, Shahzadi A, Zeitz C et al. A mutation in SLC24A1 implicated in autosomal-recessive congenital stationary night blindness. American Journal of Human Genetics 2010; 87: 523-531.
Rieder, S., S. Taourit, D. Mariat, B. Langlois and G. Gue'rin, 2001 Mutations in the agouti (ASIP), the extension (MC1R), and the brown (TYRP1) loci and their association to coat color phenotypes in horses (Equus caballus). Mamm. Genome 12: 450-455.
Sandmeyer, L., C. B. Breaux, S. Archer and B. H. Grahn, 2007 Clinical and electroretinographic characteristics of congenital stationary night blindness in the Appaloosa and the association with the leopard complex. Vet. Ophthalmol. 10: 368-375.
Schubert, G., and H. Bornshein, 1952 Beitrag zur A lyse des menschlichen Electroretinogram. Ophthalmolgica 123: 396-413.
Shen Y, Heimel J A, Kamermans M, Peachey N S, Gregg R G, Nawy S. A transient receptor potential□like channel mediates synaptic transmission in rod bipolar cells. J Neurosci. 2009; 29:6088□6093.
Silvers, W. K, 1979 The Coat Colors of Mice. Springer-Verlag, New York.
Sponenberg, D. P., and B. V. Beaver, 1983 Horse Color. Texas A&M Press, College Station, Tex.
Sponenberg, D. P., G. Carr, E. Simak and K. Schwink, 1990 The inheritance of the leopard complex of spotting patterns in horses. J. Hered. 81: 323-331.
Sponenberg, D. P., Archer, S., & Bellone, R. (2009) Patters of White with Symmetric White Patches: the Leopard Complex. In: Sponenberg D P. Equine Color Genetics. 3rd Edition. Ames, Iowa: Iowa State University Press, pp 110-121.
Steingrimsson, E., N. G. Copeland and N. A. Jenkins, 2006 Mouse coat color mutations: from fancy mice to functional genomics. Dev. Dyn. 235: 2401-2411.
Stryer, L., 1991 Visual excitation and recovery. J. Biol. Chem. 266: 10711-10714.
Sturm, R. A., R. D. Teasdale and N. F. Box, 2001 Human pigmentation genes: identification, structure and consequences of polymorphic variation. Gene 277: 49-62.
Terry, R. B., S. Archer, S. Brooks, D. Bernoco and E. Bailey, 2004 Assignment of the appaloosa coat colour gene (LP) to equine chromosome 1. Anim. Genet. 35: 134-137.
Toyofuku, K., I. Wada, R. A. Spritz and V. J. Hearing, 2001 The molecular basis of oculocutaneous albinism type 1 (OCA1): sorting failure and degradation of mutant tyrosinases results in a lack of pigmentation. Biochem. J. 355: 259-269.
van Genderen M M, Bijveld M M, Claassen Y B et al. Mutations in TRPM1 are a common cause of complete congenital stationary night blindness. American Journal of Human Genetics 2009; 85:730-736.
Vardi N. Alpha subunit of Go localizes in the dendritic tips of ON bipolar cells. Journal of Comparative Neurology 1998; 395: 43-52.
Weng K, Lu C, Daggett L P et al. Functional coupling of a human retinal metabotropic glutamate receptor (hmGlurR6) to bovine rod transduction and rat Go in an in vitro reconstitution system. Journal of Biological Chemistry 1997; 272: 33100-33104.
Witzel, D. A., J. R. Joyce and E. L. Smith, 1977a Electroretinography of congenital night blindness in an Appaloosa filly. J. Eq. Med. Surg. 1: 226-229.
Witzel D A, Riis R C, Rebhun W C et al. Night blindness in the Appaloosa: sibling occurrence. Journal of Equine Medicine and Surgery 1977; 1: 383-386.
Witzel, D. A., E. L. Smith, R. D. Wilson and G. D. Aguirre, 1978 Congenital stationary night blindness: an animal model. Invest. Ophthalmol. Vis. Sci. 1978(117): 788-793.
Xu, X. Z., F. Moebius, D. L. Gill and C. Montell, 2001 Regulation of melastatin, a TRP-related protein, through interaction with a cytoplasmic isoform. Proc. Natl. Acad. Sci. USA 98: 10692-10697.
Zeitz C, van Genderen M, Neidhardt J et al. Mutations in GRM6 cause autosomal recessive congenital stationary night blindness with a distinctive scotopic 15-Hz flicker electroretinogram. Investigative Ophthalmology and Visual Science 2005; 46: 4328-4335.
Zhiqi, S., M. H. Soltani, K. M. Bhat, N. Sangha, D. Fang et al., 2004 Human melastatin 1 (TRPM1) is regulated by MITF and produces multiple polypeptide isoforms in melanocytes and melanoma. Melanoma Res. 14: 509-516.

1. A method of screening for, diagnosing or detecting congenital stationary night blindness (CSNB) in a horse comprising determining the presence of at least one SNP allele associated with CSNB, wherein the at least one SNP allele associated with CSNB is:
a) located at position 108281765 of ECA1 and the allele associated with CSNB is C;
b) located at position 108288853 of ECA1 and the allele associated with CSNB is T; or
c) located at position 108337089 of ECA1 and the allele associated with CSNB is G.

2. The method of claim 1, comprising determining the presence of at least two of the SNP alleles associated with CSNB.

3. The method of claim 1, comprising determining the presence of all three of the SNP alleles associated with CSNB.

4. The method of claim 1, further comprising managing the CSNB if two copies of the allele associated with CSNB are detected.

5. The method of claim 1, wherein the horse is an Appaloosa, Knabstrupper, American Miniature, British Spotted Pony or Pony of the Americas.

6. A method of detecting or selecting different coat patterns in a horse comprising determining the presence of at least one SNP allele associated with leopard complex spotting (LP), wherein the at least one SNP allele associated with LP is:
a) located at position 108281765 of ECA1 and the allele associated with LP is C;
b) located at position 108288853 of ECA1 and the allele associated with LP is T; or
c) located at position 108337089 of ECA1 and the allele associated with LP is G.

7. The method of claim 6, wherein the horse is an Appaloosa, Knabstrupper, American Miniature, British Spotted Pony or Pony of the Americas.

8. The method of claim 6, comprising determining the presence of at least two of the SNP alleles associated with LP.

9. The method of claim 6, comprising determining the presence of all three of the SNP alleles associated with LP.

10. The method of claim 6, further comprising selecting a horse that has no allele associated with LP and a horse that has two copies of an allele associated with LP, and breeding the horses together to generate a horse with an LP/lp genotype.

11. The method of claim 6, further comprising selecting two horses that each have two copies of an allele associated with LP, and breeding the horses together to generate a horse with an LP/LP genotype.

12. The method of claim 6, further comprising selecting two horses that each have no allele associated with LP, and breeding the horses together to generate a horse with an lp/lp genotype.

13. The method of claim 6, further comprising selecting two horses that each have one copy of an allele associated with LP and one copy of an allele not associated with LP and breeding the horses together to generate a horse with an LP/LP, LP/lp or lp/lp genotype.

14. The method of claim 6, further comprising selecting a horse with one copy of an allele associated with LP and one copy of an allele not associated with LP and a horse with no copies of an allele associated with LP, and breeding the horses together to generate a horse with an LP/lp or lp/lp genotype.

15. The method of claim 6, further comprising selecting a horse with two copies of an allele associated with LP and a horse with one copy of an allele associated with LP and one copy of an allele not associated with LP, and breeding the horses together to generate a horse with an LP/LP or LP/lp genotype.

16. A kit for detecting a SNP allele associated with CSNB or LP comprising a probe that specifically hybridizes to a SNP allele associated with CSNB or LP and instructions for use; wherein the SNP allele associated with CSNB or LP is:
a) located at position 108281765 of ECA1 and the allele associated with CSNB or LP is C;
b) located at position 108288853 of ECA1 and the allele associated with CSNB or LP is T; or
c) located at position 108337089 of ECA1 and the allele associated with CSNB or LP is G.

17. The kit of claim 16, wherein the probe comprises the nucleotide sequence as shown in SEQ ID NO:13, 14 or 15.

18. The kit of claim 16 further comprising a second probe that specifically hybridizes to a SNP allele not associated with CSNB or LP.

19. The kit of claim 18, wherein the second probe that specifically hybridizes to a SNP allele not associated with CSNB or LP comprises SEQ ID NO:16, 17 or 18.

20. A kit for detecting a SNP allele associated with CSNB or LP comprising a pair of primers for amplifying a sequence comprising the SNP allele associated with CSNB or LP; and instructions for use; wherein the pair of primers comprise:
a) one primer upstream of the nucleotide at position 108281765 of ECA1 and one primer downstream of the nucleotide at position 108281765 of ECA1;
b) one primer upstream of the nucleotide at position 108288853 of ECA1 and one primer downstream of the nucleotide at position 108288853 of ECA1; or
c) one primer upstream of the nucleotide at position 108337089 of ECA1 and one primer downstream of the nucleotide at position 108337089 of ECA1.

21. The kit of claim 20, wherein the pair of primers is as shown in SEQ ID NOs: 1 and 2 or SEQ ID NOs: 9 and 10.

22. The kit of claim 20, wherein the pair of primers is as shown in SEQ ID NOs: 3 and 4 or SEQ ID NOs: 7 and 8.

23. The kit of claim 20, wherein the pair of primers is as shown in SEQ ID NOs: 5 and 6 or SEQ ID NOs:11 and 12.


SEQUENCE LISTING (24 sequences)

SEQ ID NO: 1 - DNA, 20 bp, Equus caballus
actggggagtctgtccactg

SEQ ID NO: 2 - DNA, 20 bp, Equus caballus
cttagctcaagccccttcct

SEQ ID NO: 3 - DNA, 20 bp, Equus caballus
cagtgaaccgggctcttaaa

SEQ ID NO: 4 - DNA, 20 bp, Equus caballus
gcagcctccaaaactgactc

SEQ ID NO: 5 - DNA, 20 bp, Equus caballus
cttaggtgaggcgaggtcag

SEQ ID NO: 6 - DNA, 20 bp, Equus caballus
acctggcaggcctatcttct

SEQ ID NO: 7 - DNA, 19 bp, Equus caballus
gcttcgtgacgctctgctt

SEQ ID NO: 8 - DNA, 22 bp, Equus caballus
ccccaaatcgtgaacttgcatt

SEQ ID NO: 9 - DNA, 24 bp, Equus caballus
cagcagggataacacttgtacagt

SEQ ID NO: 10 - DNA, 28 bp, Equus caballus
tgataagaataatgaccaggacctccat

SEQ ID NO: 11 - DNA, 22 bp, Equus caballus
tgggtacatgtcacctgcaatg

SEQ ID NO: 12 - DNA, 25 bp, Equus caballus
ttgatcaccatgggaatacattgct

SEQ ID NO: 13 - DNA, 16 bp, Equus caballus
acgtctgggttagaag

SEQ ID NO: 14 - DNA, 16 bp, Equus caballus
caaggtaccggattta

SEQ ID NO: 15 - DNA, 18 bp, Equus caballus
ctcgagactcactctgtt

SEQ ID NO: 16 - DNA, 16 bp, Equus caballus
acgtctgggttggaag

SEQ ID NO: 17 - DNA, 18 bp, Equus caballus
tgcaaggtactggattta

SEQ ID NO: 18 - DNA, 18 bp, Equus caballus
ctcgagactcaatctgtt

SEQ ID NO: 19 - DNA, 17 bp, Equus caballus
tgtcctcggccacgccc

SEQ ID NO: 20 - DNA, 18 bp, Equus caballus
gtcagcccccagccacat

SEQ ID NO: 21 - DNA, 20 bp, Equus caballus
cctgcaccgtgacttctatg

SEQ ID NO: 22 - DNA, 20 bp, Equus caballus
gccaagctggagagaacact

SEQ ID NO: 23 - DNA, 20 bp, Equus caballus
ttctctccagcttggcagtt

SEQ ID NO: 24 - DNA, 20 bp, Equus caballus
gatgctcagttcagccacaa